(19) United States
(12) Patent Application Publication
(10) Pub. No.: US 2010/0113334 A1
(43) Pub. Date: May 6, 2010

(54) METHODS FOR REDUCING OR PREVENTING TRANSMISSION OF NOSOCOMIAL PATHOGENS IN A HEALTH CARE FACILITY

(76) Inventors: Timothy S. Leach, Groton, MA (US); Daniela Jabes, Cassina Rizzardi (IT); Giorgio Mosconi, Wayne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/571,789

(22) Filed: Oct. 1, 2009

Related U.S. Application Data
(63) Continuation of application No. 12/322,420, filed on Feb. 2, 2009, now abandoned.
Application No. 12/322,420 is a continuation of application No. 10/832,965, filed on Apr. 26, 2004, now abandoned.
(60) Provisional application No. 60/465,757, filed on Apr. 25, 2003.

(51) Int. Cl. A61K 38/16; A61P 31/00

(52) U.S. Cl. 514/8

(57) ABSTRACT

The present invention provides methods and compositions useful for reducing or preventing the transmission of nosocomial pathogens or an epidemic of nosocomial pathogens in a health care facility by decolonizing the gastro-intestinal tract, skin, or nasal passage of carriers and by preventing colonization of individuals at risk who may serve as transmission vehicles or vectors to other individuals.

### RELATED APPLICATIONS

This application is a continuation of U.S. application Ser. No. 12/322,420, filed Feb. 2, 2009, which is a continuation of U.S. application Ser. No. 10/832,965, filed Apr. 26, 2004, which claims the benefit of U.S. Provisional Application No. 60/465,757, filed Apr. 25, 2003. The entire teachings of the above applications are incorporated herein by reference.

### FIELD OF THE INVENTION

This invention relates to the field of mammalian pathogenic infections.

### BACKGROUND OF THE INVENTION

Nosocomial infections are infections acquired directly or indirectly in a medical or health care setting. The highest infection rates typically occur in the intensive care units (ICUs), oncology wards and medical/surgical wards of hospitals. In recent years, the aging of the population and the practice of increasingly aggressive medical interventions have significantly contributed to the rise in the frequency and severity of nosocomial infections. The growing number of patients undergoing complex surgical procedures (e.g., transplantation of organs and foreign bodies) or being treated with immunosuppressive therapies has facilitated the transmission of nosocomial pathogens within health care settings. This is largely due to the fact that these patients, whose gastro-intestinal tract and skin harbor these pathogens, can function as transmission vehicles.

Although patients in health care facilities are especially vulnerable to infections, any individual exposed to infected patients, such as health care employees and visitors, can similarly become colonized with nosocomial pathogens. In turn, these individuals can transmit these pathogens to other patients, either by direct contact or indirectly by contaminating environmental surfaces within the facility (e.g., furniture, medical equipment, phones, or doorknobs), which then come in contact with another individual or patient. They are also at risk of becoming infected themselves.

Gram-positive bacteria are an important cause of nosocomial infections. The most common pathogenic isolates in hospitals include Enterococcus faecalis, Enterococcus faecium, Staphylococcus aureus, coagulase-negative staphylococci, and Clostridium difficile. The severity and morbidity of nosocomial infections has further been exacerbated by the emergence of variants of these strains that are resistant to many currently marketed antibiotics.

Although the prevalence and transmission of pathogens in health care settings can be minimized, for example, by frequent hand washing, cleaning, and patient isolation, the actual efficacy of such infection control measures are often limited. Thus, better strategies are needed to control the transmission of nosocomial pathogens in health care facilities.

### SUMMARY OF THE INVENTION

The present invention stems from the discovery that transmission of pathogens to uncolonized individuals may be reduced or prevented by the prophylactic administration of antibiotics. The methods of this invention may, therefore, be used to reduce the endemic rates of nosocomial infections and to prevent epidemics of these infections in healthcare facilities (e.g., hospitals, nursing homes, clinics, hospices, infirmaries, rehabilitation centers, and assisted living facilities).

Accordingly, the present invention features a method for reducing or preventing the transmission of a nosocomial pathogen by (a) identifying a carrier who is colonized with a nosocomial pathogen, and (b) administering an antibiotic, in an amount and for a duration sufficient to prevent colonization or infection by the pathogen, to a population of individuals at risk of being colonized or infected by the pathogen. Typically, the gastrointestinal tract, skin, or nasal mucosa or sinuses of the carrier is colonized with the pathogen; however, other colonization site are possible. In preferred embodiments, the carrier of the pathogen is also administered the antibiotic and the route of administration is chosen based on the colonization site. For example, the antibiotic is typically administered orally for gastrointestinal colonization, topically for dermal colonization, and intranasally for colonization of the nasal mucosa or sinuses. Antibiotic therapy is continued at least until the site is substantially decolonized, but preferably for at least an additional 7, 14, 21, or 28 days after decolonization is complete. The route of antibiotic administration to the population of individuals at risk is typically chosen based on the likely route of pathogen exposure; however, oral administration is the most common.

Suitable antibiotics for use in the methods of this invention include, for example, teicoplanin, daptomycin, oritavancin, dalbavancin, eveminomycin, quinupristin/dalfopristin, linezolid, tigecycline, colistin, amphotericin, nystatin, iseganan, ramoplanin, or any polymyxin, aminoglycoside, glycopeptide, eveminomycin, streptogramin, lipopeptide, oxazolidonone, bacteriocin, type A lantibiotic, type B lantibiotic, liposidomycin, mureidomycin, or alanoylcholine. In preferred embodiments, the antibiotic is ramoplanin which may be administered orally at a dose of 50-400 mg b.i.d., preferably, 200-400 mg b.i.d., or topically or intranasally one to six times each day in a composition consisting of 0.1% to 90% ramoplanin by weight. Preferably, substantially all of the antibiotic is non-absorbable or partially non-absorbable such that it retains antibacterial activity at the site of administration (e.g., in the lumen of the GI tract, the nasal passage, or the skin).

Carriers of nosocomial pathogens or individuals at risk include individuals who have been or will be in direct contact with a carrier, other individuals at risk, or fomites that have been in contact with a carrier or individuals at risk. Carriers of nosocomial pathogens or individuals at risk may have received broad-spectrum antibiotic therapy for at least one week within the previous month or may be immunocompromised by, for example, HIV/AIDS or an extreme of age. Other likely carriers and individuals at risk include those patients presently receiving or within 14 days of receiving chemotherapy or radiation therapy for autologous or allogeneic hematopoietic stem cell transplantation, bone marrow transplantation, solid organ transplantation, or as part of antineoplastic therapy. Individuals who are immunocompromised as a result of immunosuppressive therapy, particularly immunosuppressive steroid therapy (e.g., prednisone, dexamethasone, methylprednisolone, and hydrocortisone), administered for at least seven days are at risk for colonization. Carriers may be symptomatic or asymptomatic for the presence of the pathogen and may or may not have a bacteremia. The population of individuals at risk include patients, employees, and visitors of a health care facility, particularly individuals sharing the same floor, unit, ward, or common facilities as the carrier or an identified individual at risk. Individuals at particular risk include those that are neutropenic, immunocompromised, or at risk for developing (or diagnosed as having) enteritis, colitis, typhlitis, or mucositis of the gastrointestinal tract. Carriers may be identified by random or systematic testing. The decision to initiate preventive therapy according to the methods of this invention may be made following the identification of a carrier by random or systematic testing, or by the identification of the presence of a nosocomial pathogen on a fomite. Treatment of at risk individuals may begin prior to or without testing those individuals for colonization or infection by the nosocomial pathogen.

The methods of this invention are particularly useful for preventing the transmission of Gram-positive bacteria and particularly antibiotic-resistant Gram-positive bacteria. Such bacteria include, for example, Enterococcus spp. including E. faecium, E. faecalis, E. raffinosus, E. avium, E. hirae, E. gallinarum, E. casseliflavus, E. durans, E. malodoratus, E. mundtii, E. solitarius, and E. pseudoavium; Staphylococcus spp. including S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, and S. simulans; Streptococcus spp. including S. pyogenes, S. agalactiae, S. pneumoniae, S. bovis, and S. viridans; and clostridia: species such as C. difficile, and C. perfringens. Specifically, the methods of the present invention are effective for preventing transmission of vancomycin-resistant Enterococcus spp. (VRE), methicillin- or glycopeptide-resistant Staphylococcus spp. (e.g., MRSA, GISA, or VRSA), and penicillin-resistant Streptococcus spp. (e.g., PRSP), and C. difficile. Treatment with ramoplanin is particularly desirable if nosocomial pathogens are resistant to one or more of the following antibiotics: vancomycin, teicoplanin, daptomycin, oritavancin, dalbavancin, eveminomycin, quinupristin/dalfopristin, linezolid, or trigecycline, or alternatively one or more antibiotics belonging to the glycopeptides, eveminomycins, streptogramins, lipopeptides, oxazolidonones, bacteriocins, type A lantibiotics, type B lantibiotics, liposidomycins, mureidomycins, or alanoylcholines.

If desired, a second therapeutic agent, such as a nonabsorbable or topical antibiotic with Gram-negative activity, may be administered in combination with the ramoplanin of the invention. Exemplary antibiotics are colisitin, polymyxin B, and aminoglycosides (e.g., neomycin, amikacin, tobramycin, and gentamicin).

The invention also features a method for reducing or preventing the transmission of a nosocomial pathogen by (a) identifying a fomite that that is contaminated with a nosocomial pathogen, and (b) administering an antibiotic, in an amount and for a duration sufficient to prevent colonization or infection by the pathogen, to a population of individuals at risk of being colonized or infected by the pathogen. Fomites that may be contaminated with a nosocomial pathogen includes those that are known to have been exposed to a carrier who is colonized with a nosocomial pathogen and those that have been identified as contaminated from an unidentified source. The later category of fomites may be identified by random or systematic testing for nosocomial pathogens. Pathogen testing may involve swabbing the fomite with a biological culture swab (e.g., a cotton swab) and culturing the swab to identify the presence of a pathogen. Alternatively, pathogenic samples may be identified using molecular biological techniques such as the polymerase chain reaction (PCR) using pathogen-specific primers. Typically, preventive antibiotic therapy is orally administered; however, other routes including, for example, intranasal and dermal antibiotic administration may be used. Antibiotics suitable for reducing or preventing the transmission of the nosocomial pathogen are the same as for the previous aspect of this invention.

In one embodiment, once a fomite contaminated with a nosocomial pathogen is identified, no testing of individuals at risk is performed. Preventive antibiotic therapy is initiated immediately. Alternative, “at risk” individuals may be tested for colonization

By “broad-spectrum antibiotic” is meant an antibiotic having a wide range of activity against both Gram-positive and Gram-negative bacteria.

By “patient” is meant any human in need of medical treatment. Patients are typically institutionalized in a primary care facility such as a hospital or nursing home for example, but may also include outpatients.

By “carrier” is meant any individual in a health care facility from which a pathogen, such as a Gram-positive bacteria, can be isolated and cultured using standard techniques in the art. Carriers can be symptomatic or asymptomatic. Carriers may be, for example, patients, employees, or visitors. The pathogens that colonize a carrier may have normal antibiotic sensitivity, intermediate (reduced) antibiotic sensitivity, or the pathogen may be antibiotic-resistant.

By “exposure” is meant any contact with a carrier that can lead to the transmission of a pathogen. According to this invention, the pathogen can be transmitted by direct contact (direct physical transfer of microorganism from a carrier to an individual); indirect contact (contact of an individual with a fomite); contact with a droplet containing the pathogen that generated by coughing, sneezing, talking, and during certain procedures such as suctioning and bronchoscopy.

By “health care facility employee” is meant any individual working in any health care facility, including doctors, nurses, medical residents, medical students, emergency medical technicians, receptionists, orderlies, janitors, food service personnel, volunteers, physical therapist, visiting nurses, and administrators.

By “individual at risk” is meant any individual who may have been, has been, or will be exposed to a carrier, another individual at risk, or a fomite. Individuals at risk include individuals who are in close proximity to a carrier, and therefore include those who have shared or will share the same room, unit, ward, floor, or building as the carrier. Individuals at risk may be individuals who may not have been exposed to the carrier, but who may have been, have been, or will be exposed to another individual at risk. These individuals include, for example, visitors and health care facility employees not in direct patient contact. Thus, individuals at risk include patients in a health care facility, particularly neonatal and geriatric patients, those in intensive care units, and those that are immunocompromised (e.g., HIV/AIDS patients, neutropenic patients, and those receiving immunosuppressive chemotherapy or radiation therapy). Other individuals at risk include individuals having or at risk for developing disorders of the intestinal mucosa that impart an increased risk of developing a bacteremia (e.g., enteritis, colitis, typhlitis, or mucositis of the gastrointestinal tract). Also at are employees, visitors, and other non-patients in a health care facility. These individuals include, for example, doctor, nurse, orderly, medical student, physical therapist, health care administrator, visiting nurse, food service personnel, or janitor, and individuals working in intensive care units, oncology wards, surgical units, and geriatric wards.

By “a population of individuals at risk” is meant a plurality of individuals at risk of being colonized by a nosocomial pathogen but who are presently free from the pathogen. Populations at risk include patients, health care employees, and visitors to a health care facility.

By “health care facility” is meant any facility in which health care is provided. Medical care facilities include but are not limited to hospitals, nursing homes, clinics, hospices, infirmaries, assisted-living facilities and rehabilitation centers.

By “antibiotic-resistant Gram-positive bacteria” is meant any Gram-positive bacteria that have reduced (partially or completely) susceptibility to one or more antibiotics. Antibiotic classes to which Gram-positive bacteria develop resistance include, for example, the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylcylines (e.g., doxycycline, minocycline, tetracycline, and tigecycline), the aminoglycosides (e.g., amikacin, gentamycin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid), and the lipopeptides (e.g., daptomycin).

By “colonized” or “colonization,” as used herein, refers to a resident population of nosocomial pathogens. Colonization is frequent in the GI tract, skin, or nasal passages and may cause an infection of the carrier or be transmitted to an individual at risk. The population of the gastro-intestinal tract, skin, or nasal passage by normal GI flora, as described herein, is exemplary of what is meant by colonization. Colonization typically precedes infection, although infection does not always occur after colonization.

By “prevent colonization” is meant to reduce, inhibit, or impede the growth of a species of bacteria or other microorganism (e.g., resistant Gram-positive bacteria) such that the population of competent target pathogen in the GI tract or on the surface of the skin or nasal passages of an individual is maintained to undetectable levels using standard microbiological culture methods such as the quantification of bacterial growth from a faecal sample from a rectal swab, for example. Each of these determinations can be performed using standard microbiological techniques, such as those that conform to the standards provided by the American Society for Microbiology (Manual of Clinical Microbiology (7^{th} ed.) eds. Murray P R, Barron E J, Pfaller M A, Tenover F C, and Yolken R H, 1999, American Society for Microbiology, Washington).

By “infection” is meant an invasion and multiplication of a pathogen in body tissues, which may be clinically unapparent (asymptomatic) or result in local cellular injury (symptomatic) due to competitive metabolism, toxins, intracellular replication, or antigen antibody response. According to this invention, colonization of the colon, nasal passage, or skin is not considered to be an infection, as there is no invasion of body tissues.

“Bacteremia” is defined as the presence of bacteria in the bloodstream of a host (e.g., a patient), detectable using standard aerobic or anaerobic cultures of the blood. A patient having a bacteremia may be symptomatic or asymptomatic.

By “fomite” is meant any inanimate object or substance that is capable of transmitting infectious organisms from one individual to another. Fomites include, for example, used medical supplies such as soiled bedding, bandages, wound dressings, hypodermic needles, specula, and other medical equipment; environmental surfaces such as benchtops, tabletops, chairs, telephones, doorknobs; and used cutlery, drinking glasses, and other utensils.

“Non-absorbable” is defined as an antibiotic formulation which, when administered orally, has an absolute bioavailability of less than 10%.

By “partially non-absorbable,” when referring to an antibiotic, is meant an antibiotic formulation which, when administered orally, results in an absolute bioavailability of between 10% and 90%.

By “retains antibacterial activity” refers to a non-absorbable or partially non-absorbable antibiotic formulation which is at least 50%, 60%, 70%, 80%, 90%, 95%, or 99% bactericidal or bacteriostatic as a formulation of the same antibiotic that is more absorbable in the gastro-intestinal tract.

“Bioavailability” is defined as the fraction (F) of the orally administered dose that reaches the systemic circulation (Oates J A, Wilkinson G R. Principles of drug therapy, In Harrison's Principle of Internal Medicine (14^{th} ed.) 1998, McGraw Hill, New York.

### BRIEF DESCRIPTION OF DRAWINGS

FIG. 1 is a graph demonstrating the efficacy of oral ramoplanin treatment for decolonization of vancomycin-resistant Enterococcus (VRE) stool colonization in mice. High-density VRE colonization was established in all mice by administering orogastric VRE (day −8) in conjunction with subcutaneous clindamycin (days −10 to 0). Oral ramoplanin in drinking water (100 μg/mL or 500 μg/mL) was given for 8 days. Control mice received regular drinking water. Error bars represent SE.

### DETAILED DESCRIPTION

The present invention features methods to reduce or prevent the transmission of one or more nosocomial pathogens or infections in a health care facility. More specifically, this invention stems from our discovery that the oral, topical, or intranasal administration of an antibiotic, such as ramoplanin, in a therapeutically effective amount, alone or in combination with another antibiotic can decolonize the gastrointestinal (GI) tract, skin, or nasal passage of individuals. The GI tract, skin, and nasal passage are each known reservoirs for nosocomial pathogens in individuals (e.g., hospitalized patients), who have been exposed or will be exposed to at least one other individual whose GI tract, skin, or nasal passage is colonized by a nosocomial pathogen. Because the GI tract can serve as one of the most significant reservoirs for resistant pathogens, and because the density of skin and environmental contamination has been directly correlated with the density of contamination in the GI tract, elimination or suppression of pathogens or bacteria in the lumen of the GI tract, the skin, or the mucosal membranes of the nasal passage reduces the transmission of resistant pathogens or bacteria between carriers and individuals at risk in a health care facility. Furthermore, treating carriers and individuals at risk according to this invention also decreases contamination of fomites (e.g., environmental surfaces such as doorknobs, phones, medical equipment, and bedding) by nosocomial pathogens. Thus, according to this invention, GI, skin, or nasal passage decolonization with an antibiotic, such as ramoplanin, decreases skin and environmental contamination of nosocomial pathogens such that their transmission in health care facilities is reduced or prevented. Furthermore, decolonization of the GI tract, skin, or nasal passage also reduces the potential for the transfer of resistance genes from one pathogenic species to another, an event which typically occurs in areas characterized by high concentrations of various pathogenic strains. Therefore, this invention can also reduce the generation of new types of drug-resistant pathogenic strains.

### Flora of the Gastro-Intestinal Tract

Normally, in the upper GI tract of adult humans, the esophagus contains only the bacteria swallowed with saliva and food. The acidity of the stomach contents severely limits bacterial growth. Accordingly, the proximal small intestine has relatively limited Gram-positive flora, consisting mainly of Lactobacillus spp. and Enterococcus faecalis. Typically this region has about 10⁵-10⁷ bacteria per milliliter of luminal fluid. The distal region of the small intestine contains greater numbers of Gram-positive bacteria and other normal flora including multiple Gram-negative species (e.g., coliforms and Bacteroides). Generally, the bacterial population and diversity increases distally, reaching 10¹¹ bacteria per milliliter of faeces in the colon among which are Gram-positive bacterial species including, for example, Staphylococcus spp., Enterococcus spp., Streptococcus spp., and Clostridium spp.

Under normal conditions, the natural GI flora prevent or resist colonization by pathogenic bacterial species that may be drug resistant. Additionally, the normal flora stimulate the production of cross-reactive antibodies in the host animal, acting as antigens and inducing immunological responses. Host defense mechanisms are a complex set of humoral and cellular processes that prevent or resist microorganisms from invading the body including the bloodstream. While the normal bacterial flora are generally considered non-pathogenic in healthy individuals, these same bacteria can cause life-threatening infections if given the opportunity in patients with impaired immune function (including disruptions of normal anatomic barriers) or who are otherwise debilitated.

Traditionally, infections caused by the gastro-intestinal flora were susceptible to standard antibiotic therapy, and were thus successfully treated with known conventional antibiotics. However, with the recent emergence of stains of antibiotic-resistant bacteria, treating infections and bacteremias of this nature has become significantly more difficult. For example, VRE faecium may be resistant to all commercially available antibiotics, including linezolid and quinupristin/dalfopristin. Furthermore, patients with underlying malignancies who are colonized by VRE have rates of VRE bacteremia as high as 19%. Patients who develop bacteremias with VRE have longer hospital and ICU stays, high mortality, and greater health care costs than patients without VRE bacteremias. Thus, identification of agents that result in the suppression and/or elimination of VRE and other gastro-intestinal antibiotic-resistant Gram-positive bacteria could significantly reduce morbidity, mortality, and cost.

The highest concentrations of antibiotic-resistant bacteria, including vancomycin-resistant Enterococcus (VRE), methicillin-resistant Staphylococcus aureus (MRSA), glycopeptide intermediary susceptible Staphylococcus aureus (GISA), and penicillin-resistant Streptococcus pneumoniae (PRSP), are found in hospitals, nursing homes, and other facilities where antibiotics are heavily used. Unfortunately, these same locations also have the highest density of susceptible, at risk patients. Nosocomial infections and potential epidemics may be reduced or prevented, by decolonizing the GI tract, skin, and/or nasal passage of health care employees and visitors exposed to patients identified with antibiotic-resistant bacteria.

### Routes of Transmission

Patients with high-density stool colonization (>4 logs) are significantly more likely to contaminate the environment with VRE than those with lower density colonization (Donskey et al., N. Engl. J. Med. 343: 1925-1932, 2000). In addition to direct physical transfer of microorganisms, transmission of nosocomial pathogens, such as Gram-positive bacteria, from a carrier to an individual at risk may arise by indirect contact, involving, for example, the contact of an individual at risk with a contaminated environmental surface, such as contaminated instruments, equipment, doorknobs, bedding, furniture, clothing, or phones (i.e., fomites). Furthermore, transmission can also occur by means of droplets generated during coughing, sneezing, talking, and during certain procedures such as suctioning or bronchoscopy, or routine examination or contact with the carrier. Transmission can also occur when droplets containing microorganisms come in contact with the skin, conjunctiva, nasal mucosa, or mouth of an individual at risk. Droplet distribution involves close association, usually within one to two meters. Vehicle transmission applies to microorganisms transmitted by contaminated food, water, drugs, blood, or body fluids.

### Detection of Nosocomial Pathogens

Once a nosocomial pathogen, such as a Gram-positive bacterium, has been detected in the GI tract, on the skin, or in the nasal passages of a carrier, any patient, health care employee, or visitor who has been exposed to this patient can be immediately treated with an antibiotic (e.g., ramoplanin) therapy to reduce or prevent the transmission of the nosocomial pathogen. Nosocomial pathogens that colonize the GI tract, the skin, or the nasal passage of a patient or that cause an infection can be easily detected and characterized by a skilled artisan. For example, the Gram-positive bacteria that colonize the GI tract can be isolated, for identification and sensitivity testing, from a stool sample or culture using standard microbiological techniques. Generally, stool specimens are collected in clean (not necessarily sterile), wide-mouthed containers that can be covered with a tight-fitting lid. These containers should be free of preservatives, detergents, and metal ions and contamination with urine should also be avoided.

It is desirable that stool specimens be examined and cultured as soon as possible after collection because, as the stool specimen cools, the drop in pH soon becomes sufficient to inhibit the growth of many bacterial species. Direct microscopic examination of a faecal emulsion or stained smear to evaluate the presence of faecal pathogen forms may be valuable in the differential diagnosis of certain enteric infections. A bacterial smear for staining can also be prepared. If a delay in processing is anticipated, for example if the specimen is to be sent to a distant reference laboratory, an appropriate preservative should be used. Equal quantities of a 0.033 M sodium or potassium phosphate buffer and glycerol can be used to recover pathogenic bacteria for culturing and staining purposes.

For antibiotic sensitivity testing, a small amount of faecal specimen can be added to Gram-positive or other enrichment broth for the recovery of bacterial species. A variety of culture media containing inhibitors to the growth of normal bowel flora allows Gram-positive species to be selected. Subcultures of either isolated or mixed Gram-positive species can be prepared using antibiotic-containing culture media.

### Prevention of Transmission of Nosocomial Pathogens

According to this invention, when one carrier, or infected patient, has been identified in a health care facility, an antibiotic (such as teicoplanin, daptomycin, oritavancin, dalbavancin, eveminomycin, quinupristin/dalfopristin, linezolid, tigecycline, colistin, amphotericin, nystatin, iseganan, ramoplanin, or alternatively, a polymyxin, aminoglycoside, glycopeptide, eveminomycin, streptogramin, lipopeptide, oxazolidonone, bacteriocin, type A lantibiotic, type B lantibiotic, liposidomycin, mureidomycin, or alanoylcholine) is administered not only to the carrier, but may also be administered to one or more individuals in a population at risk. Such individuals include, for example, other patients, health care facility employees, and visitors of the health care facility. Because individuals at risk can function as transmission vehicles or vectors for nosocomial pathogens, such individuals are treated according to this invention to prevent or reduce the transmission of nosocomial pathogens. Typically, an individual at risk is any individual who has been, may have been, or will be exposed to a carrier or another individual at risk, or alternatively, any individual who may have been, has been, or will be in close proximity to a carrier or another individual at risk. Individuals at risk also include individuals who have been exposed to contaminated environmental surfaces (e.g., surfaces that have been exposed to a carrier or individual at risk, or on which a pathogen has been detected). Thus, a population at risk may include, for example, an individual who is being treated by a healthcare employee who has been, or will be exposed to at least one colonized patient, or a patient who is receiving antibiotic therapy. The prevention or reduction of epidemics and the endemic rate of nosocomial infections according to this invention can be achieved in any health care facility in which medical treatment is provided and includes, for example, hospitals, nursing homes, clinics, hospices, infirmaries, assisted-living facilities, or rehabilitation centers.

Patients

As is described herein, in addition to the infected patient who is the carrier, any patient may be administered an antibiotic such as ramoplanin to decolonize the GI tract, the skin, or the nasal passage. Preferably, any patient who has been may have been, or will be exposed to the carrier is administered ramoplanin, or another non-absorbable or partially non-absorbable antibiotic, at an effective dose to substantially decolonize, or prevent colonization of, their GI tract, skin, or nasal passage. Such patients may have been directly exposed to the carrier (by direct physical contact or by exchange of droplets), or may have indirectly been exposed by sharing common objects (e.g., phone, toilet, medical equipment, chair, bed, doorknob, etc.) or common facilities. Furthermore, these patients may also have been exposed to a carrier by the direct contact with a health care provider who is colonized or who is a carrier of the pathogen or pathogens due to recent or previous contact with a carrier or an individual at risk. Because of the difficulties in ascertaining who has come into contact with whom or what, any patient or other individual at risk who has not necessarily been exposed to the carrier but who is in close proximity to the carrier is typically treated. This will result in an entire population (e.g., individuals in the same room, ward, unit, floor, building, multiple sites or geographic area) being administered an antibiotic. Thus, a patient who may or may not have been exposed to a carrier, but may have been or will be exposed to an individual at risk (e.g., a health care employee who has been exposed to a carrier or who is in close proximity to the carrier) may be treated according to the methods of the invention. An individual at risk does not need to have been exposed to the carrier or does not need to be in close proximity to the carrier.

Patients, who are at particular risk of being infected but who may or may not have been exposed to a carrier or individual at risk, are also treated with the ramoplanin of the invention. Such patients include for example, patients hospitalized for prolonged periods of time (greater than 5 to 7 days); patients receiving systemic antibiotics (especially broad-spectrum antibiotics); immunocompromised patients; patients receiving chemotherapy or radiation therapy in preparation for autologous or allogeneic hematopoietic stem cell transplant, bone marrow transplant, or solid organ transplant; patients diagnosed as having chronic illnesses such as chronic renal insufficiency; patients having or at risk of having enteritis, colitis, or mucositis of the gastro-intestinal tract; neutropenic patients; and patients receiving or within 14 days of receiving antineoplastic radiation or chemotherapy; or patients in an ICU. Other risk factors for opportunistic infections include advanced age, organ transplantation, cancer, HIV infection, malnutrition, and other acquired or congenital causes of immune dysfunction as described supra, previous antibiotic use or surgery. Such patients are susceptible to developing bacteremia or other infections by normal GI bacteria. Likewise, disorders of the GI tract that compromise the barrier function of the GI mucosa render a patient susceptible to developing bacteremia by GI bacteria. Such conditions include, for example, colitis, proctitis, enteritis, mucositis, typhlitis, or Crohn's disease. Many of these types of conditions can be induced by therapies for other disease indications, for example, resulting from antineoplastic chemotherapy or radiotherapy, or antibiotic-induced colitis (e.g., Clostridium difficile associated diarrhea). Other patients at risk include patients with a history of bacterial infections to antibiotics.

Individuals, including health employees and visitors of the patient, who are exposed to such patients may in turn get infected, and as a result, such infections may lead to an epidemic or prevent the reduction of the endemic rate of resistant nosocomial pathogens, such Gram-positive bacteria in the health care facility.

Non-Patients at Risk

In addition to patients of the health care facility, other non-patient individuals who may be at risk for a VRE colonization of the gastrointestinal tract, skin, or nasal mucosa, include employees working in the health care facility, or visitors of patients. Health care employees include without limitation doctors, nurses, medical residents, medical students, emergency medical technicians, receptionists, orderlies, janitors, volunteers, physical therapist, visiting nurses, or administrators. Such individuals at risk are administered an antibiotic, such as ramoplanin in an amount to substantially decolonize the GI tract, the skin, or the nasal passage as they often serve as a transmission vehicle for the nosocomial pathogen, and may therefore spread the pathogen between patients within the health care facility, either directly or indirectly. Thus, non-patient individuals who are individuals at risk or carriers are treated to prevent transmission between patients, either directly or indirectly.

### Ramoplanin

Ramoplanin (A-16686; MDL 62,198; IB-777), a glycolipodepsipeptide antibiotic obtained from fermentation of Actinoplanes strain ATCC 33076, has activity against Gram-positive aerobic and anaerobic microorganisms. Ramoplanin consists of a major component (A2) and related minor components. Of these minor components, five have been structurally identified and designated as A1, A′1, A′2, A3, and A′3. Variations between structures A1, A2, and A3 are due to changes in the fatty acid moiety of ramoplanin; minor components A′2, A′2, and A′3 contain one fewer sugar residue. The term ramoplanin as used herein includes all variants of ramoplanin which may be used in a therapeutic method, or present in a pharmaceutical composition, either alone as a single component, or in any combination of two or more components.

Ramoplanin inhibits the synthesis of the bacterial cell wall by inhibiting the N-acetylglucosaminyl transferase-catalyzed conversion of lipid intermediate I to lipid intermediate II, thus interfering with peptidoglycan synthesis; this mechanism is different from that of vancomycin, teicomycin, or other cell wall-synthesis inhibitors. No evidence of cross-resistance between ramoplanin and other glycopeptides has been observed.

Ramoplanin's spectrum of activity includes staphylococci, streptococci, clostridia, enterococci, including antibiotic-resistant strains of these species (e.g., methicillin-resistant and/or glycopeptide-resistant staphylococci and vancomycin- and gentamicin-resistant enterococci). Ramoplanin is bactericidal with minimal differences between the minimum inhibitory concentration (MIC) and minimum bacteriocidal concentration (MBC) for most Gram-positive species.

In vivo, ramoplanin selectively inhibited the gram-positive colonic microflora of mice. The examples described below demonstrate that some recurrences of VRE colonization after anti-VRE treatment are due to re-expansion of small numbers of organisms that persist in the lining of the colon. VRE was detected in the cecal lining of 2 of 8 (25%) ramoplanin-treated mice that had undetectable levels of VRE in stool and cecal contents. Additionally, prior ramoplanin treatment did not facilitate the establishment of stool colonization after ingestion of VRE (FIG. 4). Previous research suggests that organisms that are able to adhere to the mucosal surfaces of the colon (i.e., epithelium or mucus layer), and are adapted to the colonic environment, may have a survival advantage over exogenously introduced organisms (Freter et al., Infect. Immunol. 39: 686-703, 1983). Minor disruption of the indigenous microflora by antibiotics (e.g., anti-VRE therapy including, for example, ramoplanin) could therefore potentially facilitate re-expansion of VRE that are already present, while being insufficient to allow overgrowth of newly introduced strains. As noted previously, anti-anaerobic antibiotics that are used concurrently with ramoplanin, or another anti-VRE therapy, may facilitate acquisition of new VRE strains after decolonization.

Ramoplanin, because of its ability to effectively suppresses VRE during treatment, can be used to reduce inter-individual cross-transmission of VRE. For example, ramoplanin treatment of all VRE-colonized patients on high-risk units (including new admissions) could markedly reduce “colonization pressure”, which plays a major role in cross-transmission (Bonten et al., Arch. Intern. Med. 158: 1127-1132, 1997).

### Dosages

To prevent infection (e.g., colonization of the gastrointestinal tract, skin, or nasal mucosa) in a patient, health care employee, or a visitor, an antibiotic, such as ramoplanin, is administered orally in an amount and for a duration sufficient to substantially decolonize the GI tract, skin, or nasal passage of nosocomial pathogens such as Gram-positive bacteria. Although the exact dosage of ramoplanin sufficient for substantially decolonizing the gastro-intestinal tract, skin, or nasal passage of a particular patient may differ, the dosage can be easily determined by a person of ordinary skill. Typically, the amount of ramoplanin that is administered is an amount that maintains the concentration of the antibiotic at least equal to the MIC for the target organism. Preferably, the amount of ramoplanin that is administered can maintain the concentration (e.g., in the stool) equivalent to two, three, four, or more times the MIC for the target organism. Thus, the particular treatment regimen may vary for each patient, dependent upon the species and resistance pattern of the identified Gram-positive bacteria, and biological factors unique to each patient including the comorbidity, disease etiology, patient age (pediatric, adult, geriatric), and the nutritional and immune status.

The suggested oral dosage of ramoplanin is at least about 50, 100, 200, 300, 400, or 500 mg/day up to as much as 600, 7000, 800, 900, or 1000 mg/day. An antibiotic may be given daily (e.g., once, twice, three times, four times, five times, six times daily, or more frequently) or less frequently (e.g., once every other day, or once or twice weekly). A suitable dose is between 50 and 400 mg, preferably 100 and 400 mg, and more preferably 200 and 400 mg BID (twice daily). The antibiotic may be contained in any appropriate amount in any suitable carrier substance, and is generally present in an amount of 1-99% by weight of the total weight of the composition. The composition is provided in a dosage form that is suitable for oral administration and delivers a therapeutically effective amount of the antibiotic to the small and large intestine, as described below.

The dosing regimen required to substantially decolonize the GI tract of nosocomial pathogens may be altered during the course of the therapy. For example, decolonization of the GI tract can be monitored periodically or at regular intervals to measure the patient's pathogenic load and dosage or frequency of antibiotic therapy can be adjusted accordingly.

Typically, therapy should last at least five days, but preferably at least one week, two weeks, three weeks, one month, two months, more than two months, or until the risk for the epidemic subsides or until the patient leaves the hospital. The antibiotic therapy should at least encompass the period during which the individual at risk is at highest risk for developing a bacteremia. More preferably, the antibiotic therapy should begin prior to exposure to a patient at risk or immediately after the exposure, and extend beyond the patient's period of highest risk. For example, a non-colonized patient who is receiving a broad-spectrum antibiotic in a setting where VRE is endemic should be treated with ramoplanin before he acquires the organism in his GI tract.

### Pharmaceutical Formulations

Pharmaceutical compositions according to the invention may be formulated to release an antibiotic substantially immediately upon administration or at any predetermined time or time period after administration. The latter types of compositions are generally known as controlled release formulations, which include formulations that create a substantially constant concentration of the drug within the GI tract over an extended period of time, and formulations that have modified release characteristics based on temporal or environmental criteria.

Antibiotic-containing formulations suitable for ingestion include, for example, a pill, capsule, tablet, emulsion, solution, suspension, syrup, or soft gelatin capsule. Additionally, the pharmaceutical formulations may be designed to provide either immediate or controlled release of the antibiotic upon reaching the target site. The selection of immediate or controlled release compositions depends upon a variety of factors including the species and antibiotic susceptibility of Gram-positive bacteria being treated and the bacteriostatic/bactericidal characteristics of the therapeutics. Methods well known in the art for making formulations are found, for example, in Remington: The Science and Practice of Pharmacy (20th ed.), ed. A. R. Gennaro, 2000, Lippincott Williams & Wilkins, Philadelphia, or in Encyclopedia of Pharmaceutical Technology, eds. J. Swarbrick and J. C. Boylan, 1988-1999, Marcel Dekker, New York. Examples of such formulations are described for example in U.S. Patent No. 60/408,596.

Ramoplanin is available as granules for oral solution, provided, for example, in packets containing 400 mg free base of ramoplanin, along with pharmaceutically acceptable excipients (e.g., mannitol, hydroxypropyl methylcellulose, magnesium stearate). The contents of the packet can be reconstituted with approximately 15-30 mL of water, and the resulting solution either consumed directly, or further diluted with water, cranberry juice, apple juice, or 7-Up prior to drinking. After consumption, the drug may be followed with subsequent amounts of these beverages or with food (e.g., cracker, bread). The 400 mg granulated powder packets are stable for at least one year at refrigerated conditions. The reconstituted ramoplanin aqueous solution has a shelf life of 48 hours when stored at refrigerated conditions. Alternatively, ramoplanin is available as capsules containing pharmaceutically acceptable excipients that are generally regarded as safe.

Topical ramoplanin may be administered to the skin or the mucus membranes of the nasal passages in an oil or water based emulsion, or as an ointment or cream in an amount ranging from 0.1% to 90% by weight, preferably less than 10% by weight. Such topical formulations may also contain pharmaceutically acceptable excipients that are generally recognized as safe (e.g., benzyl alcohol, xanthum gum, and cetomacrogol).

To decolonize the nasal passage, ramoplanin may also be administered as an aerosol. The composition is formulated (micronized) into an aerosol according to known and conventional methods for preparing such formulations. Aerosolized formulations deliver high concentrations of ramoplanin directly to the nasal passages with low systemic absorption, and include for example nasal sprays. Nasal sprays typically contain a therapeutically active ramoplanin dissolved or suspended in solution or in a mixture of excipients (e.g., preservatives, viscosity modifiers, emulsifiers, or buffering agents), in nonpressurized dispensers that deliver a metered dose of the spray.

### Example 1

### Suppression of VRE in a Mouse Model

Mice were colonized with a clinical isolate VanA strain of E. faecium (VRE) isolated from a septicemic patient. A single inoculation of 5×10⁸ cfu VRE by oral gavage (Day 0) was followed by treatment with vancomycin in the drinking water to maintain colonization. On day 22, each group received the same vancomycin-containing drinking water. One group also received ramoplanin (100 μg/mL) in its drinking water. The dose of ramoplanin per day was estimated to be 15 mg/kg, based on a standard water consumption of 150 mL/kg/day. Treatment with ramoplanin was discontinued on Day 29, and vancomycin treatment was discontinued on Day 36. The control group consisted of five mice, while the ramoplanin group consisted of four mice.

Treatment with ramoplanin significantly reduced the faecal density and carriage of VRE in mice. After one week of treatment, the VRE concentration per gram of faeces fell from 9.7 log units to an undetectable level (<3.1 log units) in all animals. Seven days after treatment with ramoplanin, the VRE concentration per gram of faeces was similar to the pre-treatment levels. The results are shown in Table 1. Table 2 further shows the in vitro activity of ramoplanin against clinically relevant Gram-positive bacteria.

### Example 2

### Efficacy of Ramoplanin for Eradication of VRE Colonization

Pathogens Studied: E. faecium C68, a previously described VanB-type clinical VRE isolate, was used for the following murine VRE experiments (Donskey et al., J. Microbial. Meth. 1807: 1-8, 2003). The minimum-inhibitory concentration of ramoplanin for VRE C68 was 0.125 μg/mL. Klebsiella pneumoniae P62 is a clinical isolate that produces an SHV type extended-spectrum β-lactamase (ESBL). Candida glabrata A239 is a clinical isolate with a fluconazole minimum-inhibitory concentration of 2 μg/mL.

Quantification of Stool Pathogens: Fresh stool specimens were processed as described by Donskey et al. (supra). In order to quantify VRE, K. pneumoniae, and C. glabrata, diluted samples were plated onto Enterococcosel agar containing vancomycin 20 μg/mL, MacConkey agar containing ceftazidime 10 μg/mL, or Sabouraud Dextrose Agar (Becton, Dickinson, and Company, Sparks, Md.) containing piperacillin/tazobactam 16 μg/mL and linezolid 8 μg/mL, respectively. The plates were incubated in room air at 37° C. far 24 or 48 hours, and the number of colony-forming units (CFU) of each pathogen per gram of sample was calculated.

High-density VRE stool colonization was established in mice by administering subcutaneous clindamycin (1.4 mg) once each day for 2 days before and 7 days after orogastric inoculation of 10⁶ colony-forming units (CFU) of VRE C68 using a stainless steel feeding tube (Perfektum, Popper & Sons, New Hyde Park, N.Y.). After discontinuation of clindamycin, mice received oral ramoplanin (100 or 500 μg/ml in drinking water) or regular drinking water (controls) for 8 days. Six mice were included in each treatment group. Stool pellets were collected every 3-4 days to monitor the density of VRE before, during, and after completion of ramoplanin treatment.

To evaluate the possibility that ramoplanin-treated mice were being re-exposed to VRE from their environment, broth-enrichment cultures for VRE were performed as previously described Ray et al. (JAMA 287: 1400-1401, 2002) after contacting cage bottoms and tops, water bottles, and food with pre-moistened cotton-tipped swabs. To evaluate whether relapses of colonization were due to persistence of VRE within the colon, 8 mice that received 8 days of oral ramoplanin treatment (100 μg/ml of water) were euthanized and portions of cecal contents and cecal lining (1×1 cm sections) were weighed, homogenized in sterile phosphate-buffered saline (PBS) using a pestle, and cultured for VRE as described above.

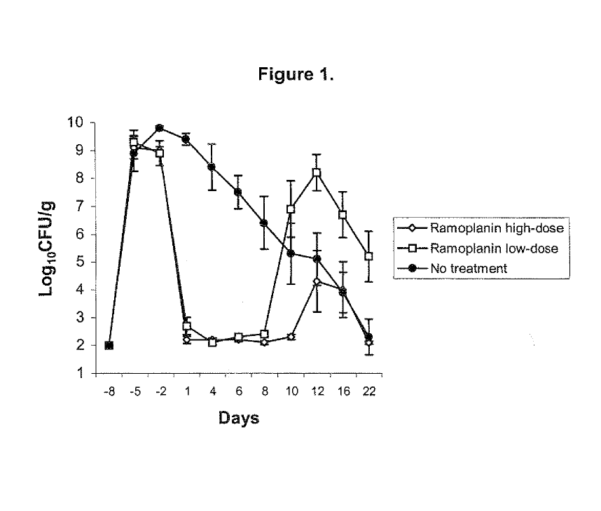

FIG. 1 shows the densities of VRE during and after completion of 8 days of ramoplanin treatment. There were no significant differences in the densities of VRE among the treatment groups prior to starting ramoplanin (days −5 and −2). All of the ramoplanin-treated mice developed undetectable levels of VRE in stool during treatment (P<0.0001 in comparison to saline controls). One hundred percent of mice receiving 100 μg/ml of ramoplanin in drinking water developed a recurrence of colonization after discontinuing treatment, whereas only 50% of mice receiving 500 μg/ml of ramoplanin developed a detectable recurrence.

During the course of ramoplanin treatment, multiple cultures of cages, food, water, and water bottles were negative for VRE. Of the 8 mice that had cultures of cecal contents and cecal lining taken on day 8 of ramoplanin (100 μg/ml drinking water) treatment, 8 (100%) had negative stool and cecal content cultures for VRE but 2 (25%) had low levels of VRE (2-3 log₁₀ CFU/g) detectable in sections of the cecal lining.

### Example 3

### Effect of Ramoplanin on the Indigenous Stool Microflora

Female CF1 mice (Harlan Sprague-Dawley, Indianapolis) weighing 25-30 g were used in these experiments. In order to minimize the risk of cross-contamination, mice were housed in individual cages with plastic filter tops. Five mice were treated with ramoplanin 100 μg/mL in drinking water for 7 days. Stool samples were collected prior to treatment, on day 7 of treatment, and 3, 6, and 11 days after discontinuation of ramoplanin. Quantitative cultures for facultative and aerobic gram-negative bacilli, enterococci, total anaerobes, Bacteroides species, Lactobacillus species, and Clostridium species were performed by plating serially-diluted specimens onto MacConkey agar (Difco Laboratories, Detroit), Enterococcosel agar (Becton Dickinson, Cockeysville, Md.), Brucella agar (Becton Dickinson), Bacteroides bile esculin agar, Rogosa agar, and Egg Yolk agar, respectively. For culture of anaerobes, stool samples were processed inside an anaerobic chamber (Coy Laboratories, Grass Lake, Mich.). Denaturing gradient gel electrophoresis (DGGE) of PCR-amplified bacterial ribosomal RNA genes from stool was performed as described by Donskey et al. (supra).

Measurement of Ramoplanin Concentrations in Stool: The concentration of ramoplanin in selected stool samples was measured using an agar well diffusion assay with Clostridium perfringens as the indicator strain (Rolfe et al., J. Infect. Dis. 147: 227-235, 1983).

The mean densities of total anaerobes and Bacteroides species were not significantly affected by seven days of ramoplanin treatment. The mean density of total facultative and aerobic gram-negative bacilli increased significantly on day 7 of ramoplanin treatment (P<0.05), but was not significantly different from baseline by 3 days after discontinuation of ramoplanin (day 10). Lactobacillus species were markedly reduced by ramoplanin treatment (P<0.001), but had returned to pre-treatment levels by 3 days after discontinuation of ramoplanin (day 10). Enterococcus species were significantly reduced by ramoplanin treatment (P<0.001), and remained significantly reduced for at least 11 days after discontinuation of ramoplanin (day 18). Ramoplanin caused relatively little disruption of the stool DGGE patterns (mean similarity indices 72% in comparison to the pre-treatment patterns). The effect of subcutaneous clindamycin, by contrast, on the DGGE patterns has mean similarity indices of 17% in comparison to pre-treatment patterns.

For the 100 μg/ml ramoplanin dose, the mean concentration in stool on day 7 of treatment was 188 μg/g of stool (range 156-312.5 μg/g; n=5 mice); no ramoplanin was detectable 3 days after discontinuation of treatment (day 10). For the 300 μg/ml dose, the mean concentration in stool on day 7 was 310 μg/ml (range 300-320 μg/g; n=5 mice).

### Example 4

### Effect of Prior Ramoplanin Treatment on the Establishment of VRE Colonization

Four hours, 1 day, 2 days, or 4 days after completing a 7-day course of oral ramoplanin (100 μg/ml in drinking water) or regular drinking water (controls), mice received orogastric inoculation of 10⁷ CFU of VRE in phosphate buffered saline. The density of VRE in stool was monitored before and 1 and 4 days after inoculation. Four mice were included in each treatment group.

Mice inoculated with 10⁷ CFU of VRE 4 hours or 1, 2, or 4 days after completion of 7 days of ramoplanin treatment did not develop significant overgrowth of VRE in comparison to controls that did not receive ramoplanin (P>0.05 for each comparison).

### Example 5

### Use of Ramoplanin to Prevent Cross-Transmission of VRE Among Mice

One set of experiments was performed to evaluate the ability of ramoplanin to prevent cross-transmission and overgrowth of VRE among mice in communal cages. High-density VRE stool colonization (˜7 log₁₀ CFU/g) was established in 2 mice as described above. Each VRE-colonized mouse was placed into a communal cage along with 4 mice with no previous exposure to antibiotics or VRE; the experimental cage was supplied with oral ramoplanin (100 μg/ml) in drinking water and the control cage was supplied with regular drinking water. All mice were treated with subcutaneous clindamycin (1.4 mg) once daily for 5 days. After 9 days, all mice were separated into individual cages and supplied with regular drinking water. The density of VRE in stool was monitored every 3-4 days during and after completion of ramoplanin treatment.

In the absence of ramoplanin treatment, VRE was rapidly transferred from one colonized mouse to 4 clindamycin-treated mice in a communal cage. With ramoplanin treatment, VRE colonization was rapidly inhibited in the colonized mouse that was added to the communal cage and none of the other mice developed detectable levels of colonization during ramoplanin treatment; after discontinuation of ramoplanin and transfer of mice to individual cages, VRE colonization was detected within 5 days in 4 of 5 mice (80%).

### Example 6

### Effect of Ramoplanin Treatment on the Establishment of Colonization by C. glabrata or K. pneumoniae

On day 2 of a 6-day course of oral ramoplanin (100 μg/ml in drinking water) or regular drinking water (controls), mice received orogastric inoculation of 10⁶ CFU of C. glabrata A239 or K. pneumoniae P62. The density of the pathogens in stool was monitored every 3-4 days. Four mice were included in each treatment group.

Ramoplanin facilitated overgrowth of K. pneumoniae P62, but not C. glabrata A239, when these pathogens were inoculated by orogastric gavage on day 2 of a 6-day course of treatment.

### Example 7

### Oral Administration of Ramoplanin to Humans

As is described in detail below, single oral doses (up to 1000 mg) and multiple oral doses (200, 400, or 800 mg BID for 10 days) of ramoplanin have been administered to healthy male volunteers. Both bioassay and HPLC-based assays to assess the absorption, distribution, metabolism, and excretion were utilized in these studies. Ramoplanin was not detected in serum/plasma or urine by either method, indicating that very little, if any, is absorbed.

### Example 7.1

### Multiple Dose Study in Healthy Male Volunteers

Healthy male volunteers were administered 200, 400, or 800 mg ramoplanin twice-a-day, for ten consecutive days. The predetermined dose was reconstituted in 5 mL water per vial, mixed with 50 mL of sweetened, aromatized solution, and immediately administered orally to the subjects.

No absorption of ramoplanin from the human gastro-intestinal tract was observed. On days 1, 5, and 10, ramoplanin was not detected in the serum at 0.5, 1, 2, 3, 6, 9, or 12 hours after the morning dose. Furthermore, no ramoplanin was detected in the urine at day 1 or 5, or in the pooled urine samples of the periods 0-12, 12-24, 24-36, 48-72, or 72-96 after the last dose.

The faecal concentrations of ramoplanin were dose related on both Day 3 (average concentration 827, 1742, 1901 μg/g in the 200, 400, and 800 mg group, respectively) and Day 10 (949, 1417, 2647 μg/g, respectively). The concentrations declined on the first day post-treatment, but remained detectable in some subjects four days post-treatment. The cumulative recovery up to Day 4 post-treatment was 25% of the administered dose.

The antibacterial activity of ramoplanin on the stool microflora was assessed in a subset of the subjects. Faecal microbial concentrations (organisms per gram of faecal matter) were determined at the following timepoints: day-4 (pre-treatment), days 4 and 10 (treatment), and days 7 and 24 (follow-up). Tolerability and absorption were also investigated.

As expected, no effect was seen in Gram-negative bacteria (enteric bacteria and Bacteroides spp.) or yeast. A marked effect was seen on Gram-positive bacteria by the first measurement on day 4. In all subjects, the concentrations of staphylococci, streptococci, and enterococci were below the level of detection by day 10.

After therapy, the gastro-intestinal tracts of the volunteers were re-colonized by normal Gram-positive bacteria. To evaluate if the predominant species that colonized the gastro-intestinal tract after therapy was that isolated before treatment, all enterococci isolated before and after ramoplanin therapy were speciated using the API system. DNA-typing was also performed when identification at the strain level was necessary. In most cases, the predominant isolate appeared to be different before and after treatment, suggesting a lack of persistence of the initial isolate.

The in vitro interaction of ramoplanin with human gastro-intestinal contents was studied. Ramoplanin was found to be microbiologically active in faeces and to bind reversibly to solid components of faeces. The binding and the subsequent release of ramoplanin from faeces would likely result in long-lasting concentrations in the gastro-intestinal tract.

### Example 7.2

### Multiple Dose Study in Asymptomatic Carriers of Gastro-Intestinal VRE

Patients identified as asymptomatic carriers of VRE were administered placebo or one of two dosages (100 mg, 400 mg) of ramoplanin BID (twice daily) for seven days. Patients were assessed by rectal swab on Days 7, 14, and 21 to determine the presence or absence of VRE. On Days 45 and 90, stool samples were analyzed for long-term effects of ramoplanin on the recurrence of, or reinfection with, VRE. All VRE isolates were tested for susceptibility to ramoplanin.

Analysis of the primary efficacy variable showed that ramoplanin effectively suppressed gastro-intestinal VRE (i.e., ramoplanin substantially decolonized the gastro-intestinal tract of VRE). None of the placebo-treated patients were VRE-free after seven days of treatment. In contrast, 17 of 21 patients (81.0%; p<0.01) who received 100 mg ramoplanin BID and 18 of 20 patients (90.0%; p<0.01) who received 400 mg ramoplanin BID were had no detectable VRE at Day 7. Seven days after cessation of treatment (Day 14), 6 of 21 patients (28.6%) who received 100 mg ramoplanin BID and 7 of 17 patients (41.2%) who received 400 mg ramoplanin BID remained VRE free. At Day 21, the number of VRE-free patients was comparable among all treatment groups.

TABLE 1
VRE suppression in a mouse model using ramoplanin

| | | | | Enterococci | |
|---|---|---|---|---|---|
| | | | | (log 10 cfu/g faeces | |
| | | | % Mice | | Total |
| Day | Study Phase | Treatment | with VRE | VRE | Enterococci |
| 22 | Prior to ramoplanin therapy | 25 mg/kg/day vancomycin | 200 | 9.7 | 9.6 |
| | | (control) | | | |
| | | 25 mg/kg/day vancomycin | 100 | 9.7 | 9.8 |
| 29 | Completion of ramoplanin | 25 mg/kg/day vancomycin | 100 | 9.4 | 9.3 |
| | therapy | (control) | | | |
| | | 25 mg/kg/day vancomycin + 15 | 0 | <3.1 | <2.4 |
| | | mg/kg/day ramoplanin | | | |
| 36 | 7 days after completion of | 25 mg/kg/day vancomycin | 100 | 9.3 | 9.6 |
| | ramoplanin therapy | (control) | | | |
| | | 25 mg/kg/day vancomycin | 100 | 8.7 | 8.6 |

TABLE 2
In vitro activity of ramoplanin against clinically
important Gram-positive bacteria*

| Organism | No. Strains Tested | Ramoplanin MIC₉₀ μg/ml |
|---|---|---|
| E faecalis | 30 | 0.5 |
| E. faecium | 10 | 0.5 |
| VRE^{†} | 235 | 0.5 |
| S. aureus (MSSA) | 140 | 0.5 |
| S. aureus (MRSA) | 100 | 0.25 |
| S. pneumoniae | 20 | <0.03 |
| Bacillus spp. | 10 | 0.25 |

*Jones RN, Barry AL. Diagn Microbiol Infect Dis 1989; 12: 279-282
^{†}Internal Phase II data: VRE faecium (n = 207), VRE faecalis (n = 26)

### Other Embodiments

All publications and patents cited in this specification are herein incorporated by reference as if each individual publication or patent was specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing the transmission of a Clostridium difficile or Clostridium perfringens pathogen, said method comprising the steps of:
a) identifying a carrier who is colonized with said pathogen; and
b) administering an antibiotic, in an amount and for a duration sufficient to reduce colonization or infection by said pathogen, to a population of individuals at risk of being colonized or infected by said pathogen from said carrier, said antibiotic being ramoplanin, wherein said population is receiving concurrent broad-spectrum antibiotic therapy.

2. The method of claim 1, wherein the gastrointestinal tract of said carrier is colonized with said pathogen and said antibiotic is orally administered to said carrier.

3. The method of claim 1, wherein said antibiotic is orally administered to said population of individuals.

4. The method of claim 1, wherein substantially all of said antibiotic is non-absorbable or partially non-absorbable when administered.

5. The method of claim 1, wherein at least one member of said population being treated is not tested for the presence of said pathogen.

6. The method of claim 1, wherein said carrier or a member of said population being treated is a patient, an employee, or a visitor in a health care facility.

7. The method of claim 1, wherein said carrier or a member of said population is a doctor, nurse, orderly, medical student, physical therapist, health care administrator, visiting nurse, food service personnel, or janitor, or works in an intensive care unit, surgical unit, or geriatric ward.

8. The method of claim 1, wherein said carrier or a member of said population is immunocompromised.

9. The method of claim 1, wherein said carrier or a member of said population has neutropenia, a human immunodeficiency virus (HIV) infection, acquired immunodeficiency syndrome (AIDS), or is within 14 days of receiving chemotherapy or radiation therapy in preparation for autologous or allogeneic hematopoietic stem cell transplant, bone marrow transplant, or solid organ transplant, or as a part of antineoplastic therapy.

10. The method of claim 8, wherein said carrier or a member of said population has been receiving immunosuppressive therapy for at least seven days.

11. The method of claim 10, wherein said immunosuppressive therapy comprises a steroid.

12. The method of claim 1, wherein a member of said population has or is at risk for enteritis, colitis, typhlitis, or mucositis of the gastro-intestinal tract.

13. The method of claim 1, wherein said pathogen is a bacterium that is antibiotic-resistant.

14. The method of claim 13, wherein said bacterium is resistant to vancomycin.

15. The method of claim 13, wherein said bacterium is resistant to one or more antibiotics selected from the group consisting of teicoplanin, daptomycin, oritavancin, dalbavancin, eveminomycin, quinupristin/dalfopristin, linezolid, tigecycline, glycopeptides, eveminomycins, streptogramins, lipopeptides, oxazolidonones, bacteriocins, type A lantibiotics, type B lantibiotics, liposidomycins, mureidomycins, and alanoylcholines.

16. The method of claim 1, wherein said ramoplanin is orally administered one to six times daily at a dosage of between about 50 mg and 400 mg.

17. The method of claim 16, wherein said ramoplanin is administered twice daily at a dosage of between about 200 mg and 400 mg.

18. The method of claim 1, wherein said population of individuals is further administered a second antibiotic having activity against Gram-negative bacteria.

19. A method for reducing the transmission of a Clostridium difficile or Clostridium perfringens pathogen, said method comprising the steps of:
a) identifying a fomite that is contaminated with said pathogen; and
b) administering an antibiotic, in an amount and for a duration sufficient to reduce colonization or infection by said pathogen, to a population of individuals at risk of being colonized or infected by said pathogen from said fomite, said antibiotic being ramoplanin, wherein said population is receiving concurrent broad-spectrum antibiotic therapy.

20. The method of claim 19, wherein said fomite has been exposed to a carrier who is colonized with said pathogen.

21. The method of claim 19, wherein said identifying step (a) comprises contacting a candidate fomite with a culture swab and culturing said swab to identify said pathogen.

22. The method of claim 19, wherein said identifying step (a) comprises the polymerase chain reaction.

23. The method of claim 19, wherein said antibiotic is orally administered to said population of individuals.

24. The method of claim 19, wherein substantially all of said antibiotic is non-absorbable or partially non-absorbable when administered.

25. The method of claim 19, wherein at least one member of said population being treated is not tested for the presence of said pathogen.

26. The method of claim 20, wherein said carrier has not been identified.

27. The method of claim 19, wherein a member of said population being treated is a patient, employee, or visitor in a health care facility.

28. The method of claim 19, wherein a member of said population being treated is a doctor, nurse, orderly, medical student, physical therapist, health care administrator, visiting nurse, food service personnel, or janitor, or works in an intensive care unit, surgical unit, or geriatric ward.

29. The method of claim 19, wherein said fomite is bedding or bandages.

30. The method of claim 19, wherein said fomite is an environmental surface.

31. The method of claim 19, wherein a member of said population is immunocompromised.

32. The method of claim 19, wherein said carrier or a member of said population has neutropenia, a human immunodeficiency virus (HIV) infection, acquired immunodeficiency syndrome (AIDS), or is within 14 days of receiving chemotherapy or radiation therapy in preparation for autologous or allogeneic hematopoietic stem cell transplant, bone marrow transplant, or solid organ transplant, or as a part of antineoplastic therapy.

33. The method of claim 31, wherein said member has been receiving immunosuppressive therapy for at least seven days.

34. The method of claim 33, wherein said immunosuppressive therapy comprises a steroid.

35. The method of claim 19, wherein a member of said population has or is at risk for enteritis, colitis, typhlitis, or mucositis of the gastro-intestinal tract.

36. The method of claim 19, wherein said ramoplanin is orally administered one to six times daily at a dosage of between about 50 mg and 400 mg.

37. The method of claim 36, wherein said ramoplanin is administered twice daily at a dosage of between about 200 mg and 400 mg.
